(19) United States
(12) Patent Application Publication
(10) Pub. No.: US 2012/0010549 A1
(43) Pub. Date: Jan. 12, 2012

(54) CERVICAL TRACTION MECHANISM

(75) Inventor: Chung-Fong Liao, Danshui Township (TW)

(73) Assignee: EXECUTIVE JET ENTERPRISE INC., TAICHUNG CITY (TW)

(21) Appl. No.: 12/831,619

(22) Filed: Jul. 7, 2010

(51) Int. Cl. A61F 5/00

(52) U.S. Cl. 602/32

(57) ABSTRACT

The present invention provides a cervical traction mechanism having a weight seat used to provide the weight force required for cervical traction. A weight adjustor is used to preset a weight force required for cervical traction as a sign of alarming. A pulling brake unit includes a pulley assembly, stay cord and traction belt joint, with one end of said pulley assembly linked to the brake unit connection end of the weight seat. A cervical traction belt has one end linked to the traction belt joint of the pulling brake unit. An alarming portion is set onto a preset location of the weight seat. When the spring weight of the weight seat reaches the weight force set by the operator, a signal is sent by the alarming portion. The cervical traction mechanism improves safety and applicability and makes it possible to avoid misoperation and recurrent damage to the patients.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention relates generally to a cervical traction mechanism, and more particularly to an innovative one which can improve the symptoms caused by neck-shoulder syndromes.

2. Description of Related Art Including Information Disclosed Under 37 CFR 1.97 and 37 CFR 1.98.

Neck-shoulder syndrome refers collectively to a variety of symptoms caused from irritation or compression against cervical nerve. This is clinically represented by neck pain, neck and shoulder muscle spasms, neck stiffness, which place restriction on the human activities, or even transmit the pain or numbness to the scapular port, upper shoulder, forearm and fingers. The neck-shoulder syndrome in youth is mainly originated from improper posture, which places the cervical intervertebral disc under stress and leads to further backward slippage for oppressing and stimulating the endorrhachis and epidural root. The neck-shoulder syndrome in middle-aged and elderly persons is mainly originated from nerve oppression caused by cervical spine degeneration and bone spurs.

Generally, a weight type cervical traction mechanism is used to improve the symptoms arising from neck-shoulder syndrome. Said weight type cervical traction mechanism is provided with a main spring, a stay cord and a pulling rope. The stay cord is pulled to make the main spring generate tractive force, however, the weight type cervical traction mechanism lacks an adjustment and alarming system that signals a warning to the users while improving the tractive force. In such a case, recurrent damage may occur due to any negligence, so there is still room for improvement through innovation.

Thus, to overcome the aforementioned problems of the prior art, it would be an advancement if the art to provide an improved structure that can significantly improve the efficacy.

Therefore, the inventor has provided the present invention of practicability after deliberate experimentation and evaluation based on years of experience in the production, development and design of related products.

### BRIEF SUMMARY OF THE INVENTION

The enhanced efficacy of the present invention is as follows:

Based on the unique configuration of the present invention wherein the “cervical traction mechanism” mainly comprises: a weight seat, a weight adjustor, a pulling brake unit, a cervical traction belt and an alarming portion. The cervical traction mechanism is operated in such a manner that the weight adjustor can preset the weight force required for cervical traction, and permits the alarming portion to send out an alarming signal for the operator once the spring weight of the weight seat reaches the weight force set by the operator. This can help avoid misoperation and recurrent damage to the patient, thus meeting diversified neck-shoulder syndromes by improving substantially the safety and applicability for the sake of different patients.

Although the invention has been explained in relation to its preferred embodiment, it is to be understood that many other possible modifications and variations can be made without departing from the spirit and scope of the invention as hereinafter claimed.

### BRIEF DESCRIPTION OF THE SEVERAL VIEWS OF THE DRAWINGS

FIG. 1 shows an exploded perspective view of the preferred embodiment of the present invention.

FIG. 2 shows an assembled perspective view of the preferred embodiment of the present invention.

FIG. 3 shows a sectional view of the weight seat of the present invention.

FIG. 4 shows a schematic view of the weight seat of the present invention.

FIG. 5 shows a partially enlarged view of FIG. 4.

FIG. 6 shows a schematic view of another preferred embodiment of the alarming portion of the present invention.

FIG. 7 shows: a schematic view showing the operating status of the preferred embodiment of the present invention.

FIG. 8 shows a schematic view of the present invention wherein the pulley assembly is provided with a releaser.

### DETAILED DESCRIPTION OF THE INVENTION

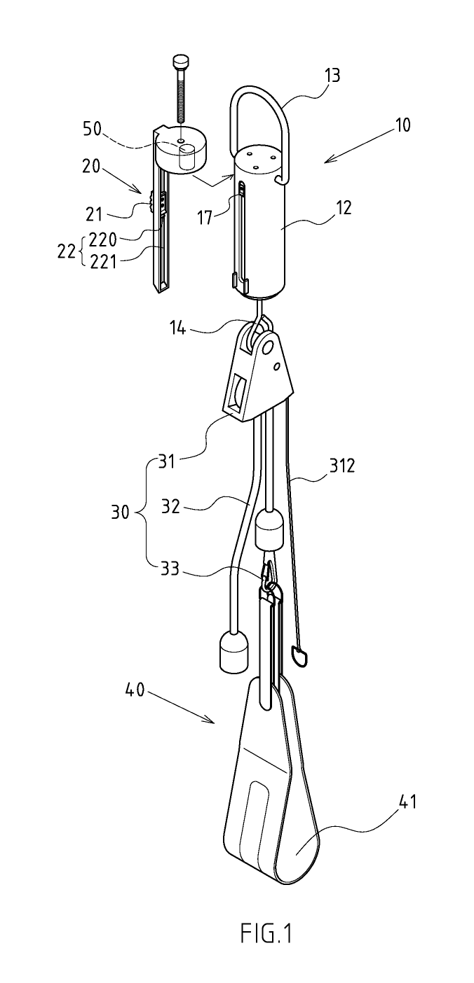

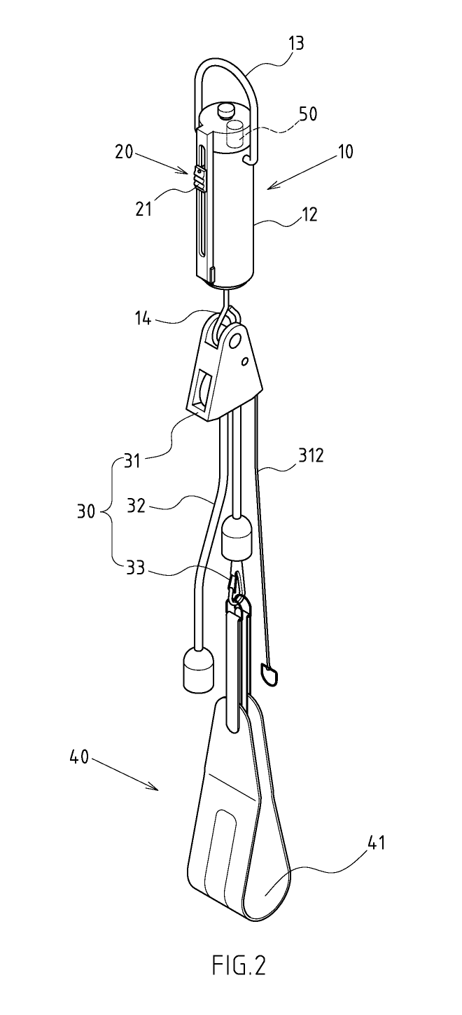

FIGS. 1-3 depict preferred embodiments of a cervical traction mechanism of the present invention, which, however, are provided for only explanatory objective for patent claims. Said cervical traction mechanism includes a weight seat 10, comprising of an inner seat 11 mated with an outer seat 12 to define a fixed end 13, a brake unit connection end 14, and a holding space 15 within the inner seat 11. A spring weight 16 is assembled into said holding space 15 to provide the weighting force required for cervical traction. A trigger element 17 is formed laterally onto the weight seat 10.

A weight adjustor 20 is set laterally onto the weight seat 10; the weight adjustor 20 comprises of an adjustment switch 21 and an adjustment brake 22. Said weight adjustor 20 is used to preset a weight force required for cervical traction as a sign of alarming.

A pulling brake unit 30 includes a pulley assembly 31, a stay cord 32 and a traction belt joint 33. One end of said pulley assembly 31 is linked to the brake unit connection end 14 of the weight seat 10, and the stay cord 32 is pulled to control the weight force of the spring weight 16 on the weight seat 10. Said stay cord 32 is wound onto the pulley assembly 31, and pulled to haul the spring weight 16 for obtaining the weight force.

A cervical traction belt 40 has one end linked to the traction belt joint 33 of the pulling brake unit 30. The cervical traction belt 40 is provided with a lower jaw support 41.

An alarming portion 50 is set onto a preset location of the weight seat 10. When the spring weight 16 of the weight seat 10 reaches the weight force set by the operator, a signal is sent by the alarming portion 50.

Of which, the adjustment brake 22 comprises of an electrical contact spring 220 and a regulating circuit board 221. When the trigger element 17 of the weight seat 10 is abutted onto the electrical contact spring 220, the preset weight force is reached once the electrical contact spring 220 touches the regulating circuit board 221.

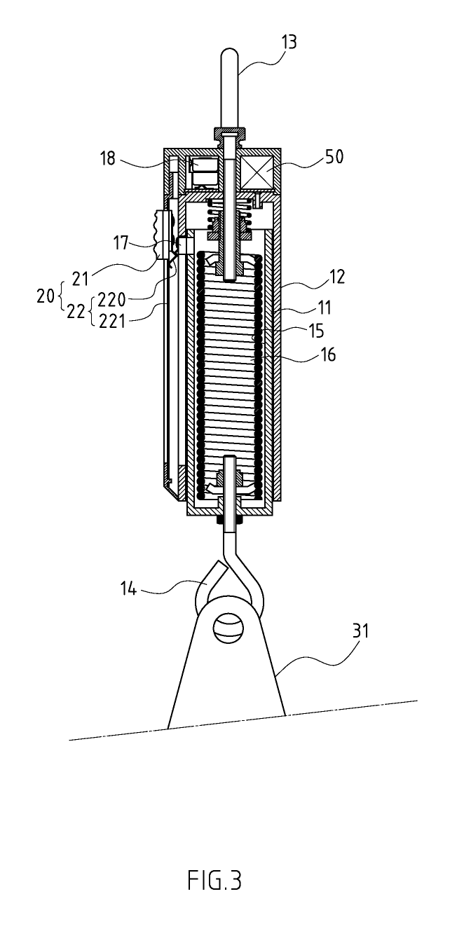

Of which, the weight seat 10 is provided with a battery 18 (marked in FIG. 3), which provides the power source for alarming portion 50.

The alarming portion 50 is a buzzer, a luminous element or a combination thereof.

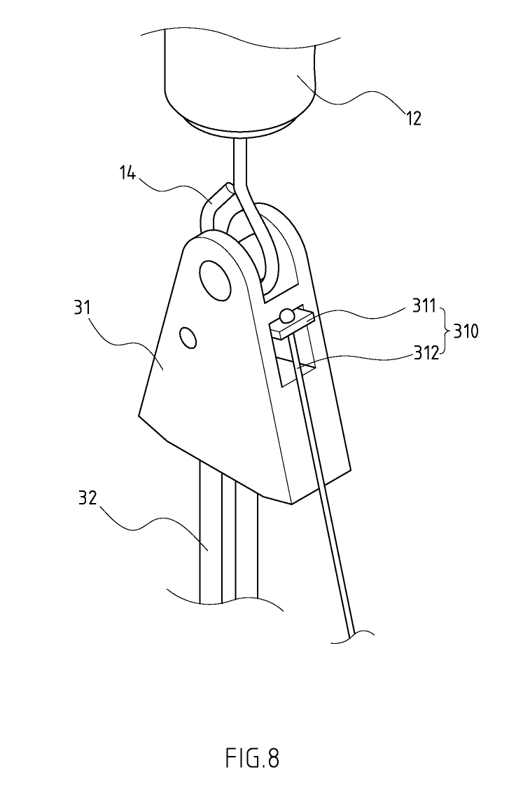

The pulley assembly 31 is provided with a releaser 310 (marked in FIG. 8). The releaser 310 comprises of a brake disc 311 and a release rope 312. When the tractive force is extremely large, users may just pull the release rope 312, enabling the brake disc 311 to release the pulling state of the stay cord 32 for avoiding damage.

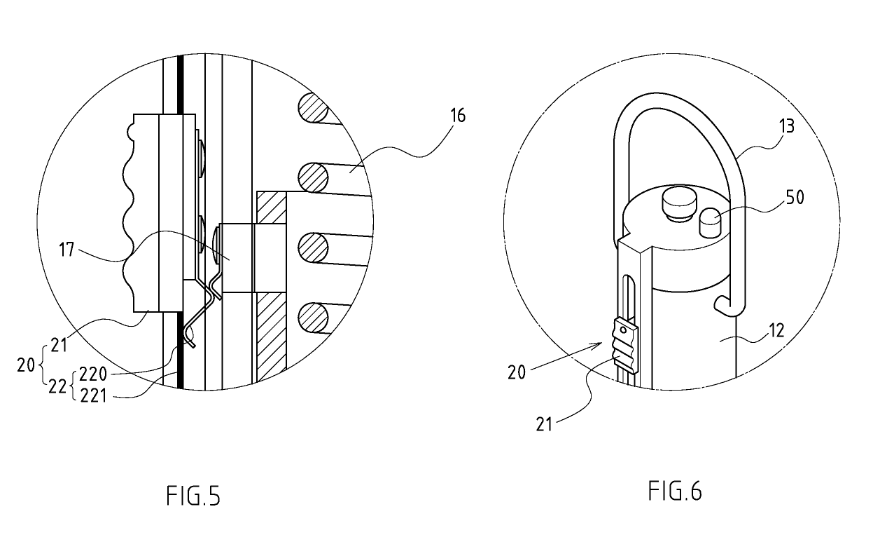

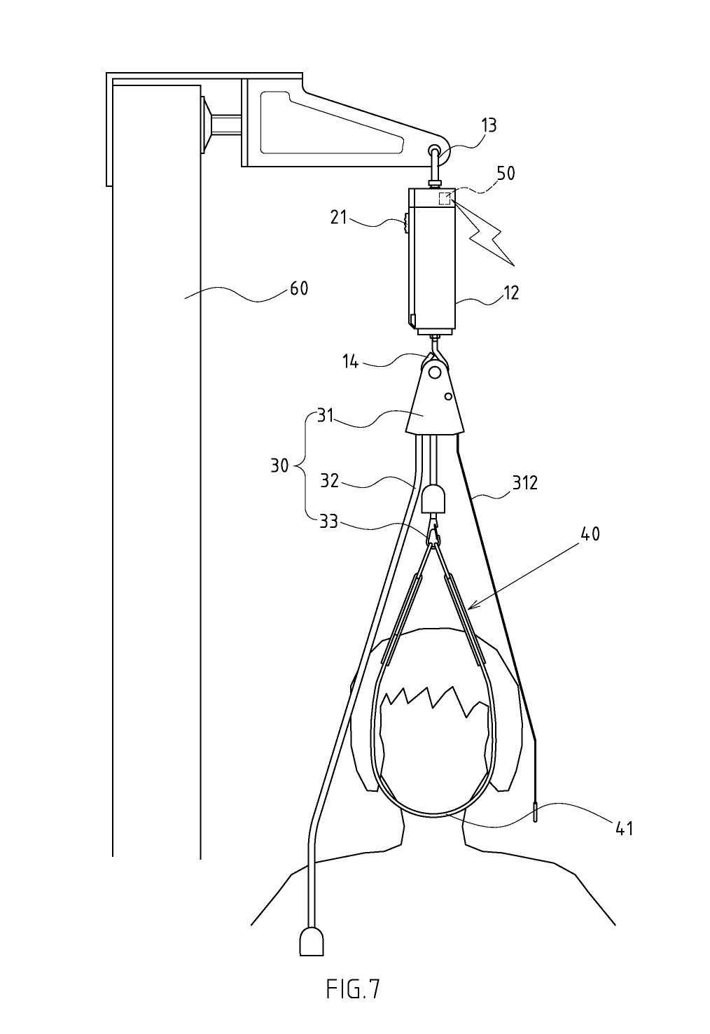

Based upon above-specified structural configuration, one of the innovative features of the cervical traction mechanism of the present invention lies in that the weight seat 10 of the cervical traction mechanism is provided with said alarming portion 50. The alarming portion 50 can send out a signal for the operator to avoid misoperation and recurrent damage when the weight seat 10 reaches the weight force set by the operator. The alarming portion 50 can be implemented in several patterns, for instance, as shown in FIG. 7, the alarming portion 50 is a buzzer that can provide an acoustic alarming to the operator. Or, as shown in FIG. 6, the alarming portion 50 is a luminous element that can projects beams of light to the deaf.

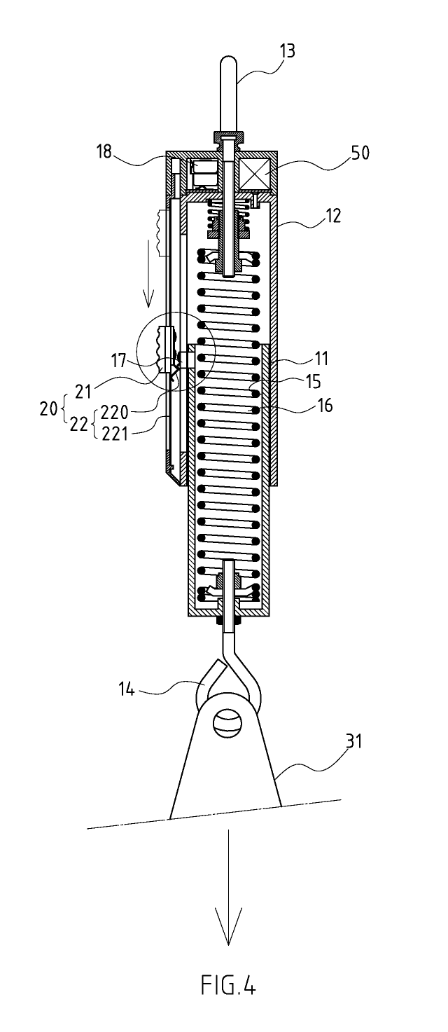

When the cervical traction mechanism of the present invention is applied, as shown in FIG. 7, the fixed end 13 of the weight seat 10 shall be mounted onto the door panel 60, then the adjustment switch 21 of the weight adjustor 20 is finetuned so that it can be dropped to the required weight force under pressure. Meanwhile, the electrical contact spring 220 is driven downwards. When the lower jaw of the patient is fixed onto the lower jaw support 41 of the cervical traction belt 40, the stay cord 32 can be pulled to haul the spring weight 16 such that the trigger element 17 can slide together with the inner seat 11 (disclosed in FIG. 4). When the trigger element 17 is abutted onto the electrical contact spring 220, the preset weight force is reached once the electrical contact spring 220 touches the regulating circuit board 221 (disclosed in FIG. 5). In such a case, the alarming portion 50 sends out a signal to the operator, and the cervical traction mechanism permits to make adjustment according to the individual weight, i.e. up by 1 kg every 2 days, and a dwell time of 15-20 m every time, thereby increasing intervertebral space and foramen of spinal cord and nerve root, mitigating or even eliminating the stimulation and oppression against the nerve root.

I claim:

1. A cervical traction mechanism, comprising:
a weight seat, comprising of an inner seat mated with an outer seat to define a fixed end, a brake unit connection end, a holding space within the inner seat; a spring weight is assembled into said holding space to provide the weighting force required for cervical traction; and a trigger element is formed laterally onto the weight seat;
a weight adjustor, set laterally onto the weight seat; the weight adjustor comprises of an adjustment switch and an adjustment brake; said weight adjustor is used to preset a weight force required for cervical traction as a sign of alarming;
a pulling brake unit, comprising of a pulley assembly, a stay cord and a traction belt joint; one end of said pulley assembly is linked to the brake unit connection end of the weight seat, and the stay cord is pulled to control the weight force of the spring weight on the weight seat;
a cervical traction belt, with one end linked to the traction belt joint of the pulling brake unit; and the cervical traction belt is provided with a lower jaw support;
an alarming portion, set onto a preset location of the weight seat; when the spring weight of the weight seat reaches the weight force set by the operator, a signal is sent by the alarming portion.

2. The structure defined in claim 1, wherein said adjustment brake of the weight adjustor comprises of an electrical contact spring and a regulating circuit board; when the trigger element of the weight seat is abutted onto the electrical contact spring, the preset weight force is reached once the electrical contact spring touches the regulating circuit board.

3. The structure defined in claim 1, wherein the weight seat is provided with a battery that provides the power source for alarming portion.

4. The structure defined in claim 1, wherein the alarming portion is a buzzer, a luminous element or a combination.

5. The structure defined in claim 1, wherein the pulley assembly is provided with a releaser; the releaser comprises of a brake disc and a release rope.
